(19) United States
(12) Patent Application Publication
(10) Pub. No.: US 2012/0269848 A1
(43) Pub. Date: Oct. 25, 2012

(54) COMPOSITIONS AND METHODS FOR TREATING AND PREVENTING INFECTION

(76) Inventors: George A. Scheele, La Jolla, CA (US); James E. Hildreth, Woodstock, MD (US)

(21) Appl. No.: 13/526,365

(22) Filed: Jun. 18, 2012

Related U.S. Application Data
(63) Continuation of application No. 12/554,521, filed on Sep. 4, 2009, now abandoned.

(51) Int. Cl. A61K 39/12; A61K 39/245; A61K 39/29; A61K 39/145; A61K 39/155; A61K 39/275; A61K 39/215; A61K 39/295; A61P 31/12; A61P 31/22; A61P 31/18; A61P 31/14; A61K 31/724; A61P 31/04; A61P 31/10; A61P 33/02; A61K 35/12; A61K 35/14; A61P 37/00; A61P 31/16; A61K 39/21

(52) U.S. Cl. 424/202.1; 424/204.1; 424/208.1; 424/229.1; 424/225.1; 424/209.1; 424/211.1; 424/232.1; 424/221.1; 424/207.1; 514/58; 424/184.1; 424/529

(57) ABSTRACT

This invention relates to cholesterol-sequestering agents and methods of using cholesterol-sequestering agents to treat or prevent infection. The compositions of the invention can be used in vitro or in vivo to decrease the load of a microorganism in a biological sample. Methods of generating an immune response against a microorganism are also included.

### CROSS REFERENCE TO RELATED APPLICATIONS

This application is a continuation of U.S. patent application Ser. No. 12/554,521, filed Sep. 4, 2009, which claims priority from U.S. patent application Ser. No. 10/625,090, filed Jul. 22, 2003, which claims priority from U.S. Provisional Application No. 60/400,333, filed Jul. 22, 2002. The prior applications are incorporated herein by reference in their entirety.

### STATEMENT AS TO FEDERALLY SPONSORED RESEARCH

This invention was made with Government support under grant numbers HD-40772, AI-31806, and HD-39613 awarded by the National Institutes of Health. The Government may have certain rights in the invention.

### TECHNICAL FIELD

This invention relates to methods and compositions for treating and preventing infection, and more particularly to methods and compositions using cholesterol-sequestering agents.

### BACKGROUND

Therapeutic approaches have proven largely ineffective for treating infections with envelope viruses such as human immunodeficiency virus (HIV) and other sexually transmitted viral diseases (STDs). In addition, non-sexually transmitted viral diseases such as influenza as well as infections caused by a variety of other microorganisms continue to flourish largely unchecked. The medical community is thus faced with a major need to develop: viricides that destroy human immunodeficiency virus (HIV), herpes simplex virus (HSV) and other causative agents of sexually transmitted diseases (STDs); viricides that destroy non-sexually transmitted viruses causing diseases such as influenza and parainfluenza; and compounds that destroy wide spectrum of infectious microorganisms, such as viruses, bacteria, mycobacteria, fungi, and protozoa.

### SUMMARY

In one aspect, the invention features a method of preparing a pharmaceutical composition. The method includes the steps of: contacting in vitro a sample containing at least one envelope virus with an amount of a cholesterol-sequestering agent effective to lyse the envelope virus, thereby resulting in a lysate; and formulating at least a portion of the lysate in a pharmaceutical composition suitable for administration to a mammal, wherein the pharmaceutical composition contains an amount of the lysate sufficient to generate an immune response against the envelope virus when administered to the mammal.

A “cholesterol-sequestering agent” refers to a compound that binds to cholesterol and extracts and depletes cholesterol from a biological membrane such as a plasma membrane or a membrane of an envelope virus. A cholesterol-sequestering agent preferentially extracts cholesterol from lipid rafts present in a biological membrane. The cholesterol-sequestering agent can be, for example, a cyclodextrin. In one example, the cholesterol-sequestering agent is a beta-cyclodextrin such as 2-OH-propyl-beta-cyclodextrin.

An “immune response against an envelope virus” refers to an immune response directed against an epitope of an intact envelope virus or an antigenic portion of an envelope virus. The immune response can be a cellular and/or a humoral immune response. For example, the immune response can be directed against a portion of a viral peptide presented by a virally infected cell in the context of a major histocompatibility complex molecule.

The envelope virus can be, for example, a human immunodeficiency virus (HIV) such as HIV-1 or HTV-2; a human herpes virus (HHV) such as HHV1, HHV2, HHV3, HHV4, HHV5, HHV6, HHV7, or HHV8; a hepatitis virus such as hepatitis B virus, hepatitis C virus, or hepatitis D virus; a pox virus such as a small pox virus or molluscum contagiosum virus; an orthomyxovirus such as an influenza virus types A, B, or C; a paramyxovirus such as a mumps virus or a parainfluenza virus type 1, 2, 3, or 4; a human T-cell lymphotropic (HTLV) virus such as HTLV type I or II; a togaviruses such as rubella virus, yellow fever virus, or sinbis virus; ebola virus; or a coronavirus such as severe acute respiratory syndrome (SARS) virus. The envelope virus can be any type or any strain of a given envelope virus. Non-limiting examples of envelope viruses and various types are described herein.

In one embodiment, the sample contains a plurality of different envelope viruses, a plurality of different types of a given envelope virus, and/or a plurality of different strains of a given type of envelope virus. Accordingly, the pharmaceutical composition can be used to generate an immune response in the mammal against a plurality of different viruses and/or strains of a given virus. Such a composition can be particularly useful when treating an individual infected or suspected of having been infected by a plurality of different viruses.

The pharmaceutical composition can be formulated for any route of administration.

In one example, the pharmaceutical composition is formulated for oral administration. In such an embodiment, the pharmaceutical composition can optionally contain an enteric coating. In another example, the pharmaceutical composition is formulated for intravenous administration. In another example, the pharmaceutical composition is formulated for intramuscular administration. In another example, the pharmaceutical composition is formulated for subcutaneous, intradermal, inhalation, rectal, vaginal, conjunctival, or otic administration.

In another aspect, the invention features a pharmaceutical composition containing a cholesterol-sequestering agent and at least a portion of a lysate of an envelope virus, wherein the composition is suitable for administration to a mammal and comprises an amount of the lysate sufficient to generate an immune response against the envelope virus when administered to the mammal. Such a pharmaceutical composition can be generated by contacting in vitro a sample containing at least one envelope virus with an amount of a cholesterol-sequestering agent effective to lyse the envelope virus, as described herein.

The cholesterol-sequestering agent can be any of the compounds described herein. The cholesterol-sequestering agent can be, for example, a cyclodextrin. In one example, the cholesterol-sequestering agent is a beta-cyclodextrin such as 2-OH-propyl-beta-cyclodextrin.

The pharmaceutical composition can be formulated for any route of administration, as detailed herein.

In one example, the pharmaceutical composition is formulated as a solid dosage form, e.g., an enteric coated solid dosage form.

In another aspect, the invention features a method of generating an immune response in a mammal by administering to a mammal an amount of a pharmaceutical composition described herein effective to generate an immune response against an envelope virus in the mammal.

The method can also include an additional step of administering to the mammal an amount of a cholesterol lowering agent effective to reduce the level of serum cholesterol in the mammal. A “cholesterol lowering agent” refers to a compound that inhibits the synthesis of cholesterol. A cholesterol lowering agent can optionally bind to HMG-CoA reductase (HMGR), an enzyme that catalyzes a key step in cholesterol production, and block a catalytic region of HMGR that participates in cholesterol synthesis. Examples of cholesterol lowering agents include, for example, members of the statin family, e.g., lovastatin (Mevacor), fluvastatin (Lescol), pravastatin (Pravachol), simvastatin (Zocor), cervastatin (Baycol), atorvastatin (Lipitor), compactin (also known as mevastatin), and rosuvastatin (Crestor).

In another aspect, the invention features a method of treating a viral infection in a mammal. The method includes the steps of: selecting a mammal infected by an envelope virus or suspected of having been infected by an envelope virus; and administering to the mammal an amount of a cholesterol-sequestering agent effective to reduce viral load in the mammal.

The cholesterol-sequestering agent can be any of the compounds described herein, e.g., a beta-cyclodextrin such as 2-OH-propyl-beta-cyclodextrin.

In one embodiment, the amount of the cholesterol-sequestering agent administered to the mammal is effective to reduce viral load in the blood of the mammal.

In another embodiment, the amount of the cholesterol-sequestering agent administered to the mammal is effective to reduce viral load in an interstitial space of the mammal.

The method can include an additional step of administering to the mammal an amount of a cholesterol lowering agent effective to reduce the level of serum cholesterol in the mammal.

The cholesterol-sequestering agent can be administered by any route of administration, as detailed herein. For example, the cholesterol-sequestering agent can be administered intravenously. An intravenous administration of the cholesterol-sequestering agent can be, for example, by a bolus injection or by infusion.

For an infusion, the cholesterol-sequestering agent can be infused into the mammal over a period of at least 1, 2, 3, 4, 5, 6, 7, 8, 9, 10, 15, 20, or 30 minutes.

For an infusion, the cholesterol-sequestering agent can be administered in at least 2, 3, 4, 5, 6, 7, 8, 9, 10, or more intravenous administrations separated by an interval of at least 30 minutes, 1, 2, 3, 4, or more hours. In some embodiments, the infusions are separated by at least 12 hours, one day, one week, or longer.

In one embodiment, the cholesterol-sequestering agent is co-administered with at least one antimicrobial agent, e.g., antiviral agent. For example, for methods that treat a mammal infected with HIV, a nucleoside reverse transcriptase inhibitor, a non-nucleoside reverse transcriptase inhibitor, a protease inhibitor, a fusion inhibitor, and/or an integrase inhibitor can also be administered to the mammal.

In one example, the method includes a step of measuring the titer of the envelope virus before and/or after administration of the cholesterol-sequestering agent.

In another example, the method includes a step of measuring an immune response in the mammal against the envelope virus before and/or after administration of the cholesterol-sequestering agent.

In one embodiment, the cholesterol-sequestering agent is administered to a dermal surface of the mammal.

For example, where a mammal has a skin lesion resulting from an infection by the envelope virus, the cholesterol-sequestering agent can be applied topically to the skin lesion. In such an embodiment, the topical administration of the cholesterol-sequestering agent can result in a reduction in viral load in the skin lesion. Examples of envelope virus infections that can be treated by such a method include but are not limited to a herpes virus (e.g., HHV1 or HHV2 for the treatment of Herpes labialis and Herpes genitalis) and a pox virus infection.

In some embodiments, the cholesterol-sequestering agent is administered to the dermal surface in the form of a cream. A “cream” refers to a semisolid emulsion of either the oil-in-water or the water-in-oil type, formulated for topical use.

In some embodiments, the cholesterol-sequestering agent is co-administered with at least one antiviral agent.

In another aspect, the invention features a method of treating or preventing an infection in a mammal. The method includes the steps of: selecting a mammal infected by a microorganism or suspected of having been infected by a microorganism, wherein during at least a portion of its life cycle the microorganism enters a cell of the mammal by endocytosis; and administering to the mammal an amount of a cholesterol-sequestering agent effective to reduce the load of the microorganism in the mammal.

The microorganism can be, for example, a bacterium (e.g., anthrax or chlamydia), a mycobacterium (e.g., mycobacterium tuberculosis), a virus (e.g., an envelope virus or a non-envelope virus, e.g., a protein coated virus such as picorna virus), a fungus, or a protozoan.

The cholesterol-sequestering agent can be administered by any route of administration, as detailed herein. For example the cholesterol-sequestering agent can be administered by inhalation or by intrathecal administration.

The cholesterol-sequestering agent can be administered as a respiratory prophylactic for an infection of the lungs, e.g. an influenza virus or a mycobacterium tuberculosis infection.

In one embodiment, the cholesterol-sequestering agent is administered to the upper respiratory tract of the mammal.

In another embodiment, the cholesterol-sequestering agent is administered to the lower respiratory tract of the mammal.

In another aspect, the invention features a method of generating an immune response in a mammal. The method includes the steps of: contacting a population of lymphocytes in vitro with an amount of the pharmaceutical composition of claim 18 effective to generate an immune response against an envelope virus, thereby resulting in activated lymphocytes; and administering the activated lymphocytes to a mammal.

In one embodiment, the population of lymphocytes is derived from the mammal prior to contacting with the pharmaceutical composition.

In another embodiment, the population of lymphocytes is derived from a second mammal prior to contacting with the pharmaceutical composition.

In another aspect, the invention features a method of treating a viral infection in a mammal. The method includes the steps of: removing blood from a mammal infected by an envelope virus; contacting the blood with an amount of a cholesterol-sequestering agent effective to reduce viral load in the blood, thereby resulting in reduced-viral load blood; and administering the reduced-viral load blood to the mammal.

In one embodiment, the blood of the mammal is perfused from a first blood vessel of the mammal, through an extracorporeal apparatus fluidly connected to the first vessel, wherein the extracorporeal apparatus adds the cholesterol-sequestering agent to the blood, and is reintroduced to the mammal in a second blood vessel that is fluidly connected to the extracorporeal apparatus.

The method can include an additional step of removing all or a portion of the cholesterol-sequestering agent from the reduced-viral load blood prior to administering the reduced-viral load blood to the mammal.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### DETAILED DESCRIPTION

The present invention provides methods and compositions for treating or preventing an infection by a microorganism. The methods and compositions of the invention make use of a cholesterol-sequestering agent that can have one or more of several possible effects on a microorganism. For some microorganisms such as envelope viruses, a cholesterol-sequestering agent may cause the lysis of the virus. By removing cholesterol from a viral membrane, a cholesterol-sequestering agent not only disrupts the ordered structure of membrane elements, but further destroys the integrity of the membrane itself leading to disruption of the viral membrane and leakage of viral contents, an irreversible process that fully inactivates the viral particle. Accordingly, a cholesterol-sequestering agent can cause a direct reduction in viral load in a biological sample. In some instances, a cholesterol-sequestering agent may block the uptake of an intracellular pathogen by blocking endocytosis in a cell. By such a mechanism, the cholesterol-sequestering agent can prevent the infection of the cell. In other instances, a cholesterol-sequestering agent can block the fusion of a microorganism with the plasma membrane and/or block the budding of the microorganism from lipid rafts on the membrane of the infected cell.

### Cholesterol-Sequestering Agent

Any cholesterol-sequestering agent can be used in the methods and compositions of the invention. As described herein, a cholesterol-sequestering agent binds to cholesterol and extracts and depletes cholesterol from a biological membrane, such as a plasma membrane or a membrane of an envelope virus. A cholesterol-sequestering agent preferentially extracts cholesterol from lipid rafts present in a biological membrane.

Examples of cholesterol-sequestering agents include compounds such as cyclodextrins, nystatin, and filipin. Cyclodextrins include both naturally occurring cyclodextrins, e.g., alpha, beta, and gamma cyclodextrins, as well as derivatives of naturally occurring cyclodextrins. Non-limiting examples of derivatives of naturally occurring cyclodextrins include derivatives of beta cyclodextrin such as hydroxypropyl beta cyclodextrin, carboxy-methyl beta cyclodextrin, and methyl beta cyclodextrin. For a detailed description on cyclodextrins and derivatives thereof, see, e.g., Parrish, M. A. “Cyclodextrins—a Review.” Sterling Organics Ltd., Newcastle-Upon-Tyne, England; and cyclodex.com.

Beta cyclodextrin, a simple sugar ring structure containing seven alpha (1-4) glucopyranose units, has the ability to rapidly extract cholesterol from lipid rafts, thereby disrupting their ordered membrane structure. As a result of cholesterol removal, lipid rafts are dispersed in the plane of the membrane and the mechanisms responsible for entry and exit of envelop viruses from target cells are abolished.

Beta cyclodextrin has a particularly high affinity for cholesterol. When used at concentrations ranging from 5-100 mM, 2-HP-BCD removes membrane cholesterol within minutes. At the molecular level, beta cyclodextrin resembles a toroid or cup-like structure with openings at both the top and bottom. The toroid structure contains hydrophilic groups on the exterior surface and hydrophobic groups on the interior surface. The hydrophylic groups confer solubility in aqueous medium while the hydrophobic groups form the hydrophobic pocket that binds the cholesterol.

Hydroxypropyl beta cyclodextrin is an example of a derivative of beta cyclodextrin that can be used in the methods of the invention. Hydroxypropyl beta cyclodextrin is a partially substituted poly(hydroxpropyl) ether of beta cyclodextrin. The basic closed circular structure of beta cyclodextrin is maintained in hydroxypropyl beta cyclodextrin. The glycosidic oxygen forming the bond between the adjacent glucose monomers and the hydrogen atoms lining the cavity of the cyclodextrin impart an electron density and hydrophobic character to the cavity. Organic compounds interact with the walls of the cavity to form inclusion complexes. The hydroxyl groups and the hydroxypropyl groups are on the exterior of the molecule and interact with water to provide the increased aqueous solubility of the hydroxypropyl beta cyclodextrin and the complexes made with the hydroxypropyl beta cyclodextrin. For a detailed description of the structure of hydroxypropyl beta cyclodextrin, see, e.g., Muller et al. (1986) “Hydroxypropyl-B-cyclodextrin derivatives: Influence of average degree of substitution on complexing ability and surface activity” J. Pharm. Sci. 75.

### Pharmaceutical Compositions

A cholesterol-sequestering agent can be used to lyse an envelope virus, thereby resulting in a viral lysate. As detailed herein, by removing cholesterol from the membrane of an envelope virus, a cholesterol-sequestering agent not only disrupts the ordered structure of the membrane elements, but further destroys the integrity of the membrane itself leading to disruption of viral membranes and leakage of viral contents into the medium, an irreversible process that can fully inactivate the viral particle. All or a portion of the viral lysate can be used to formulate a pharmaceutical composition that, when administered to a mammal, generates an immune response against the virus in the mammal.

In some embodiments of the invention, a cholesterol-sequestering agent is administered to a mammal in an amount effective to reduce the load of an envelope virus in the mammal. Accordingly, a pharmaceutical composition can be formulated to contain a cholesterol-sequestering agent described herein.

Envelope viruses that can be used in to generate a pharmaceutical composition include, but are not limited to: a human immunodeficiency virus (HIV) such as HIV-1 or HF/-2; a human herpes virus (HHV) such as HHV1, HHV2, HHV3, HHV4, HHV5, HHV6, HHV7, or HHV8; a hepatitis virus such as hepatitis B virus, hepatitis C virus, or hepatitis D virus; a pox virus such as a small pox virus or molluscum contagiosum virus; an orthomyxovirus such as an influenza virus types A, B, or C; a paramyxovirus such as a mumps virus or a parainfluenza virus type 1, 2, 3, or 4; a human T-cell lymphotropic virus (HTLV) such as HTLV type I or II; a togaviruses such as rubella virus, yellow fever virus, or sinbis virus; ebola virus; or a coronavirus such as severe acute respiratory syndrome (SARS) virus.

A pharmaceutical composition of the invention can be used to treat or prevent any clinical condition that results from an infection by en envelope virus, including but not limited to AIDS (HIV infection), certain cancers (caused by HTLV types I and II), fever blisters or cold sores (Herpes labialis; HHV1 infection), genital herpes (Herpes genitalis; HHV2 infection), chicken pox (HHV3 infection), herpes zoster or shingles (HHV3 infection), mononucleosis (HHV4 infection), cytomegalovirus infection (HHV infection), Kaposi's Sarcoma (HHV8 infection), German measles (rubella virus infection), or severe acute respiratory syndrome (SARS virus infection).

A viral lysate prepared as described herein can be incorporated into a pharmaceutical composition. The pharmaceutical composition can optionally also include the cholesterol-sequestering agent used to prepare the viral lysate. In some embodiments, all or a portion of the cholesterol-sequestering agent is removed from the lysate before or during the preparation of the pharmaceutical composition. For example, the pharmaceutical composition can be formulated to ensure that the amount of the cholesterol-sequestering agent contained therein has reduced or absent toxicity.

A pharmaceutical composition typically includes a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, intramuscular, vaginal, rectal, conjunctival, otic, or intrathecal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the viral lysate (and optionally the cholesterol-sequestering agent) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the viral lysate (and optionally the cholesterol-sequestering agent) is delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The viral lysate (and optionally the cholesterol-sequestering agent) can also be prepared in the form of suppositories (e.g., with conventional suppository bases such as cocoa butter and other glycerides) or retention enemas for rectal delivery.

In one embodiment, the viral lysate (and optionally the cholesterol-sequestering agent) is prepared with carriers that will protect the lysate against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system, if possible, that targets such compounds to the site of affected tissue in order to minimize potential damage to uninfected cells and, thereby, reduce side effects.

The pharmaceutical composition can be administered in a single administration or in multiple administrations. For example, the pharmaceutical composition can be administered about one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a cholesterol-sequestering agent can include a single treatment or, preferably, can include a series of treatments.

The pharmaceutical compositions can be included in a container, pack, or dispenser together with instructions for administration.

### Methods of Treatment

The invention includes methods of treating an infection, e.g., a viral infection, in a mammal infected by an envelope virus or suspected of having been infected by an envelope virus, by administering to the mammal an amount of a cholesterol-sequestering agent effective to reduce the load of the infecting microorganism in the mammal.

The invention includes methods of treating or preventing an infection in mammal by administering a viral lysate as described herein to a mammal. A pharmaceutical composition containing the viral lysate can be administered in 1, 2, 3, or more administrations. An adjuvant can optionally be administered with the pharmaceutical composition.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The invention includes methods of treating or preventing an infection in a mammal infected by a microorganism or suspected of having been infected by a microorganism that, during at least a portion of its life cycle enters a cell of the mammal by endocytosis, by administering to the mammal an amount of a cholesterol-sequestering agent effective to reduce the load of the microorganism in the mammal. The microorganism can be, for example, a bacterium (e.g., anthrax or chlamydia), a mycobacterium (e.g., mycobacterium tuberculosis), a virus (e.g., an envelope virus or a non-envelope virus, e.g., a protein coated virus such as picorna virus), a fungus, or a protozoan.

### Antimicrobial Compounds

The methods described herein to treat or prevent an infection can be used in combination with one or more anti-microbial agents. For example, an HIV infection can be treated with a pharmaceutical composition described herein (containing a viral lysate and/or a cholesterol-sequestering agent) and a co-administration of a nucleoside reverse transcriptase inhibitor, a non-nucleoside reverse transcriptase inhibitor, a protease inhibitor, a fusion inhibitor, and/or an integrase inhibitor can also be administered to the mammal. A bacterial infection can optionally be treated with a pharmaceutical composition described herein and a co-administration of an antibiotic.

### Extracorporeal Methods

The invention also includes methods of using an extracorporeal apparatus to treat a viral infection in a mammal. According to such as method, blood is removed from a mammal infected by an envelope virus and is contacted with an amount of a cholesterol-sequestering agent effective to reduce viral load in the blood. Following the ex vivo reduction of viral load in the blood of the mammal, the reduced-viral load blood is returned to the mammal.

The extracorporeal apparatus used to reduce viral load in the blood of the mammal is fluidly connected to a first blood vessel of the mammal, from which blood flows into the apparatus. The extracorporeal apparatus contains a cholesterol-sequestering agent that is contacted with the blood of the mammal while in the extracorporeal apparatus. After treatment of the blood with the cholesterol-sequestering agent, the blood is reintroduced to the mammal in a second blood vessel that is fluidly connected to the extracorporeal apparatus.

The extracorporeal apparatus can optionally be prepared along the lines of machines that are used for kidney dialysis. The extracorporeal apparatus can, for example, contain a multiplex of hollow fiber membranes whereby the blood from the mammal can flow through the lumen and the dialysis fluid surrounds the hollow fibers. The mammal's blood can be treated in the extracorporeal apparatus with a cholesterol-sequestering agent such as 2-HP-BCD by admixing 2-HP-BCD to the blood contained in the machine, for a period of time, such that free viral particles, e.g., HIV particles, are lysed. Because 2-HP-BCD is small in molecular weight, it can be added to the dialysis solution. 2-HP-BCD can be removed from the blood by passing the decontaminated blood through another series of hollow fiber membranes, whereby the 2-HP-BCD with and without cholesterol will pass through the membrane into the dialysis fluid. In this case there would be no BCD in the dialysis fluid.

The mammal's blood can be returned to the mammal, devoid or reduced in BCD and converted to disrupted or destroyed viral particles, from the machine to the patient. HIV-infected lymphocytes can also be removed from the blood using a 10-20 micron filter. Lymphocytes are approximately 15-20 microns in diameter whereas red blood cells are only 7 microns in diameter.

As part of an extracorporeal treatment, viral titer within the plasma and/or within white blood cells (lymphocytes, mononuclear cells and macrophages) can be analyzed, e.g., by using PCR for HIV nucleotide sequences. PCR could be used both before and after the procedure. Extracorporeal treatment can be conducted in combination with other methods such as UV irradiation of viral particles or nucleotide crosslinking procedures or other inactivation procedures.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of preparing a pharmaceutical composition, the method comprising:
contacting in vitro a sample comprising at least one envelope virus with an amount of a cholesterol-sequestering agent effective to lyse the envelope virus, thereby resulting in a lysate; and
formulating at least a portion of the lysate in a pharmaceutical composition suitable for administration to a mammal, wherein the pharmaceutical composition comprises an amount of the lysate sufficient to generate an immune response against the envelope virus when administered to the mammal.

2. The method of claim 1, wherein the cholesterol-sequestering agent is a cyclodextrin.

3. The method of claim 2, wherein the cyclodextrin is a beta-cyclodextrin.

4. The method of claim 3, wherein the beta-cyclodextrin is 2-OH-propyl-beta-cyclodextrin.

5. The method of claim 1, wherein the envelope virus is a human immunodeficiency virus (HIV), a human herpes virus a hepatitis virus, a pox virus, an influenza virus, a parainfluenza virus, a human T-cell lymphotropic virus (HTLV), or a coronavirus.

6. The method of claim 1, wherein the sample comprises a plurality of different envelope viruses.

7. A pharmaceutical composition comprising a cholesterol-sequestering agent and at least a portion of a lysate of an envelope virus, wherein the composition is suitable for administration to a mammal and comprises an amount of the lysate sufficient to generate an immune response against the envelope virus when administered to the mammal.

8. The pharmaceutical composition of claim 7, wherein the cholesterol-sequestering agent is a cyclodextrin.

9. The pharmaceutical composition of claim 8, wherein the cyclodextrin is a beta-cyclodextrin.

10. The pharmaceutical composition of claim 9, wherein the beta-cyclodextrin is 2-OH-propyl-beta-cyclodextrin.

11. A method of generating an immune response in a mammal, the method comprising administering to a mammal an amount of the pharmaceutical composition of claim 7 effective to generate an immune response against an envelope virus in the mammal.

12. The method of claim 11, further comprising administering to the mammal an amount of a cholesterol lowering agent effective to reduce the level of serum cholesterol in the mammal.

13. A method of treating a viral infection in a mammal, the method comprising:
selecting a mammal infected by an envelope virus or suspected of having been infected by an envelope virus; and
administering to the mammal an amount of a cholesterol-sequestering agent effective to reduce viral load in the mammal.

14. The method of claim 13, wherein the cholesterol-sequestering agent is a cyclodextrin.

15. The method of claim 14, wherein the cyclodextrin is a beta-cyclodextrin.

16. The method of claim 15, wherein the beta-cyclodextrin is 2-OH-propyl-beta-cyclodextrin.

17. The method of claim 13, wherein the cholesterol-sequestering agent is co-administered with at least one antiviral agent.

18. The method of claim 13, wherein the envelope virus is a herpes virus.

19. The method of claim 18, wherein the herpes virus is human herpes virus 1 or human herpes virus 2.

20. The method of claim 13, wherein the envelope virus is a poxvirus.

21. A method of treating or preventing an infection in a mammal, the method comprising:
selecting a mammal infected by a microorganism or suspected of having been infected by a microorganism, wherein during at least a portion of its life cycle the microorganism enters a cell of the mammal by endocytosis; and
administering to the mammal an amount of a cholesterol-sequestering agent effective to reduce the load of the microorganism in the mammal.

22. The method of claim 21, wherein the microorganism is a bacterium, a mycobacterium, a virus, a fungus or a protozoan.

23. The method of claim 21, wherein the a cholesterol-sequestering agent is administered with a polyol.

24. The method of claim 23, wherein the polyol is polyethylene glycol.

25. A method of generating an immune response in a mammal, the method comprising:
contacting a population of lymphocytes in vitro with an amount of the pharmaceutical composition of claim 19 effective to generate an immune response against an envelope virus, thereby resulting in activated lymphocytes; and
administering the activated lymphocytes to a mammal.

26. The method of claim 25, wherein the population of lymphocytes is derived from the mammal prior to contacting with the pharmaceutical composition.

27. The method of claim 25, wherein the population of lymphocytes is derived from a second mammal prior to contacting with the pharmaceutical composition.

28. A method of treating a viral infection in a mammal, the method comprising:
removing blood from a mammal infected by an envelope virus;
contacting the blood with an amount of a cholesterol-sequestering agent effective to reduce viral load in the blood, thereby resulting in reduced-viral load blood; and
administering the reduced-viral load blood to the mammal.

29. The method of claim 28, wherein the blood of the mammal is perfused from a first blood vessel of the mammal, through an extracorporeal apparatus fluidly connected to the first vessel, wherein the extracorporeal apparatus adds the cholesterol-sequestering agent to the blood, and is reintroduced to the mammal in a second blood vessel that is fluidly connected to the extracorporeal apparatus.

30. The method of claim 28, further comprising removing all or a portion of the cholesterol-sequestering agent from the reduced-viral load blood prior to administering the reduced-viral load blood to the mammal.
